(19) United States
(12) Patent Application Publication
(10) Pub. No.: US 2006/0149300 A1
(43) Pub. Date: Jul. 6, 2006

(54) Vasectomy tool

(76) Inventors: John W. Jessen, Seattle, WA (US); Richard E. Berger, Seattle, WA (US)

Correspondence Address: PATRICK M. DWYER PC, SUITE 114, 1818 WESTLAKE AVENUE N, SEATTLE, WA, 98109, US

(21) Appl. No.: 11/256,516

(22) Filed: Oct. 22, 2005

Related U.S. Application Data
(60) Provisional application No. 60/621,405, filed on Oct. 22, 2004.
(60) Provisional application No. 60/647,377, filed on Jan. 26, 2005.

(51) Int. Cl. A61B 17/32

(52) U.S. Cl. 606/167

(57) ABSTRACT

A vasectomy tool with a reamer operatively engaged upon a pull rod. The pull rod is extendable into a releasably locked position against spring tension and the reamer has a series of cutting disks on a shaft, the shaft having a blunt conical tip. The tool also has a handle, and the extendable pull rod and reamer are disposed within the handle. The handle has a catch release button interengageable with a spring catch release on the pull rod, and also has an integral forceps mechanism disposed on an outside of the handle.

This application claims priority to U.S. Provisional Application 60/621,405 filed Oct. 22, 2004 and 60/647,377 filed Jan. 26, 2005.

### TECHNICAL FIELD

The invention relates to method and apparatus for secure vasectomy; more particularly, it relates to a vasectomy tool and a method of using same.

### BACKGROUND OF THE INVENTION

Current state of the art in vasectomy procedure is to transect (with optional disposal of a small section) the vas differens (“vas”) and then burn cauterize the body end of the vas to seal it. The problem (resulting in an estimated 8% failure rate for this procedure, where the vas rejoins and at least partial fertility is restored) is that the burned end is subject to necrosis, physical separation and reabsorption into the body, leaving the highly regenerative vas ends free to come into contact with each other and rejoin. What is needed is a procedure and device or kit for transecting the vas and scarring cut vas ends permanently so that nothing short of a surgical stent will ever restore fertility.

Vasectomy is the most effective form of birth control and the only permanent method of male birth control. No-scalpel vasectomy technique has provided a safer and easier method for approaching the vas during vasectomy and has increased its popularity. Yet the actual interruption of the vas is accomplished by numerous techniques, most of which have not been well studied in terms of efficacy. Vasectomy failure occurs in up to 9% of cases studied, and typically results from sperm leakage from the transected ends of the vas. Sperm then create an inflammatory reaction in the tissue and “find” the other end of the vas. A transient or permanent restructuring of the vas may then occur, allowing sperm to get into the semen. A number of ways have been used in an attempt to prevent sperm from successfully jumping the gap and causing the vasectomy to fail.

In particular, the method consisting of just taking out a piece of the vas and creating a gap is often unsuccessful and accounts for the largest number of vasectomy failures. Tying the cut ends with suture, after removing a piece of the vas, is the most common method worldwide. It is not technically challenging, and can be taught to ancillary medical personnel. Unfortunately, if the suture is tied too tightly, the end of the vas loses its blood supply, necroses, and falls off, as discussed above. This opens the way to recanalization, and in one study, 12% of such cases did recanalize.

In a ‘facial interposition’ method, a piece of the vas is removed, the ends are tied, and the body end of the vas is fixed in a separate tissue plane. It has the same shortcomings discussed above, however if sperm leak out they have a more difficult journey to the body end of the vas. This is not only a more difficult procedure, requiring extensive training, but the failure rate is still around 5%.

The most apparently effective method currently being used, apart from what is disclosed herein, is intravasal electro-cautery of mucosa, with a piece of the vas removed. An electro cautery unit with a hot wire is placed into the cut ends of the vas, destroying the mucosa or inner lining by heat. Scarring takes place inside the vas while the outside muscular layer remains viable. The scarring causes an effective sealing of the vas ends. The procedure requires expensive and specialized equipment, such as a disposable electro cautery unit—prohibitively expensive in many parts of the world. If a unipolar unit is used, the vas may be too extensively scarred and make later reversal impossible. Also the length of time the electro cautery is applied and the intensity of heat must be precisely controlled, for too much heat will destroy the entire end, lead to necrosis and the kind of leakage discussed above; too little results in ineffective scarring and no sperm seal.

There are no known devices that eliminate the inner mucosal surface of the vas without electrical means. What is needed is a device and method that only affects the mucosa of the vas, and optimally, a small amount of the muscle layer, so that there is greatly reduced chance of necrosis. What is needed is a method and tool that can be used worldwide that is simple with minimal tissue dissection, and can be used in areas where finances and or power supply irregularities do not permit electro cautery. What is needed is a device that can be either disposable, single use, or resterilizable, with readily available pre-sterilized kits.

### DISCLOSURE OF THE INVENTION

What is here presented is such a device and method that only affects the epithelial layer of the vas, the mucosa of the vas, and optimally, a small amount of the muscle layer, so that there is greatly reduced chance of necrosis. It is a method and tool that can be used worldwide that is simple with minimal tissue dissection, and can be used in areas where finances and or power supply irregularities do not permit electro cautery. It is a device that can be either disposable, single use, or resterilizable, with readily available pre-sterilized kits.

A vasectomy tool with a reamer operatively engaged upon a pull rod is disclosed. The pull rod is extendable into a releasably locked position against spring tension and the reamer has a series of cutting disks on a shaft, the shaft having a blunt conical tip. The tool also has a handle, and the extendable pull rod and reamer are disposed within the handle. The handle has a catch release button interengageable with a spring catch release on the pull rod, and also desirably has an integral vas-clamping forceps mechanism disposed on an outside of the handle.

In general what is further disclosed is a probe-like reamer that has a series of cutting disks, or an array of concentric conical cutters, on a shaft, and the shaft has a radiused or blunt conical tip. The cutting disks have generally circumferential cutting edges. Preferred cutting disks have a generally frustoconical leading surface and either a generally back-cut back surface, or a generally straight or planar back surface, with the frusto conical leading surface and the back surface defining at least in part the circumferential cutting edge. The conical tip advantageously dilates the highly compliant vas tissue about twice its normal inner diameter, so that the cutters or cutting disks slide into place within the lumen of a partially transected vas, and the cutting edges readily engage the vas tissue.

A preferred tool has the reamer removably engaged upon the pull rod. In one embodiment, the pull rod terminates in a collet, and the reamer terminates, at an end opposite the conical tip, in a chuck end sized to releasably engage the collet.

In one embodiment of the vasectomy tool, a reamer is operatively engaged upon a pull rod, the pull rod is extendable into a releasably locked position against spring tension from a spring. The tool advantageously has a handle like that of a retractable pen, and the extendable pull rod and reamer are disposed within the handle. The handle advantageously has a catch release button interengageable with a spring catch release on the pull rod. Optionally, the handle has an integral forceps mechanism disposed on an outside of the handle, and cooperates with the handle to grip the vas while the inserted reamer is removed quickly under spring tension. In general it is believed that the inertia of the vas, even when not firmly gripped by forceps or fingers, and the sudden accelerated motion of the reamer withdrawing from the vas, causes the cutting edges to engage the wall of the vas lumen and cut, either relatively cleanly with a back-cut kind of blade, or bluntly with tearing with a blade that has a straight or planar back. The uniform, and selectably variable (with optional selection of different spring rate springs for any particular tool) extraction force eliminates technique variables and allows all operators to achieve repeatable and optimal results.

Preferred embodiments of the tool have most if not all of the cutting disks spaced from one another along the shaft at a distance approximately the same as a diameter of the cutting disks which in preferred embodiments have uniformly sized diameters. The cutting disks may advantageously be spaced from one another along the shaft at a distance between 60% and 110% of a diameter of a disk, with around 90% of a diameter of a disk (+/−3 to 5%) being a particularly well adapted spacing. One tool disclosed has cutting disks that are approximately 1.1 millimeters in diameter and spaced from one another by approximately 1 millimeter. In an alternate injection molded plastic embodiment, the cutting disks are spaced from one another along the shaft at a distance approximately ⅔ of a diameter of a disk.

The optional forceps preferably terminate in a pair of generally hemi-cylindrical jaws, the jaws extending past a body of the handle so that they are disposed to grip and overlap a vas section around the reamer when it is an extended and locked position. The forceps jaws advantageously each have notches that are complimentary and overlapping with respect of one jaw to the other.

The reamer cuts or tears off its relatively precise volume of tissue, including lining mucosa and a thin layer of muscle, and pulls it inwardly toward the central reamer shaft and carries it out as the reamer is extracted. Spacing of cutting disks can be optimized for tissue removing effectiveness of the cutter array. In general if they are too closely spaced, the epithelium can not be squeezed in between the cutting disks to engage the cutting edges, and the area between disks is too small to remove sufficient tissue to induce the scarring response to the tissue injury. If they are too far apart, the inter-disk spaces become clogged with too much tissue and the cutting action can be halted or interfered with before all the epithelium between each pair of disks is effectively removed. In general optimal spacing is as disclosed herein.

A preferred kit would comprise such a tool or a set of them, if disposable, and a set of reamers if reusable with a double ended carrier/installation tool, along with the usual cutting blade, clamps, and surgical accessories appropriate to the procedure.

Also disclosed is a vasectomy method, with the steps of 1) sliding into a vas opening a multi-bladed reamer; 2) holding the vas against the inserted reamer; 3) quickly pulling the reamer out of the vas to remove at least a portion of vas mucosal layer and some of the vas muscle layer tissue; whereby scarification of the vas is induced, and the vas is sealed by resultant scarring. The reamer is optimally sized to dilate the vas into which it is inserted, and preferred cutting edges of the cutting disks are adapted for tearing rather than cutting tissue as the reamer is removed quickly. In preferred methods, the reamer has the spring-loaded pull bar feature, and the pull bar and attached reamer are pulled out of the vas under selectable spring tension. Preferably, the reamer and pull bar are disposed within a spring-loaded handle that includes a vas-gripping forceps mechanism, and the step of holding the vas is accomplished with the forceps, and the step of quickly pulling out the reamer is accomplished with the release of the spring-loaded pull bar. The reamer is preferably removably attachable to the pull bar, and before and after the steps of sliding in the reamer and quickly removing the reamer, a removable reamer is respectively attached to the pull bar, and removed from the pull bar, with the steps of attaching and removing the removable reamer being effected with a holder tool, preferably double ended, for inserting and removing the reamer into and out of the pull bar.

For optimizing results with an optimal amount of tissue repeatably removed from the vas lumen during each procedure, irrespective of size variations which might cause a reamer to fit more or less tightly, a clamping forceps is advantageously provided. The forceps compresses the vas generally circumferentially, urging the mucosa into the spaces between the cutters on the reamer before the reamer withdrawal spring is released to extract the reamer. A tissue stop is now also desirably provided which prevents the rearward motion, or elongation, of the vas as the reamer pulls it back during extraction.

After insertion of the reamer, the operator squeezes the forceps sufficiently to just close the clamp around the vas, and then continues to squeeze further to further compress or bow the arms of the forceps to push against the button on the tool handle, whereby the spring catch protruding from the casing of the pull rod is pressed and the spring is released to pull the reamer back.

The optional notches on the rim of the jaws can capture a larger diameter vas and compress it against the reamer before the surfaces of the opposing jaws come into contact. The notches of the two jaws overlap as they come together and the width of the opposing arches gets smaller; thus the tissue of smaller diameter vasi is also compressed circumferentially until the jaws come into full contact. The gentle spring pressure of the long forceps arms applies uniform clamping force on all size vasi and this assures uniform success.

A low-cost disposable version of the reamer can be produced by plastic injection molding if the profile of the cutting disks is modified to eliminate undercuts inherent in the alternate lathe-turned steel design. The resulting chisel edges chop into the vas lumen mucosa as the applicator's clamping forceps squeeze the vas. The multiple segments are thus held firmly between the disks as the spring snaps the reamer back. It is believed this action causes the captive mucosa to delaminate from the soft smooth muscle which makes up the body of the vas and be carried out by the reamer. This configuration desirably produces a rougher surface on the inside of the vas. This, in combination with the bruising effect of the clamping forceps, facilitates the formation of a robust occlusive scar.

Optimal separation between the cutting flutes in this configuration is approximately equal to two-thirds of the diameter of the reamer. This allows enough space between the cutters for the mucosa to drape over the edges and at the same time produces the smallest possible sections, which enables easy delamination and tissue removal.

The optional double-ended cylindrical changing tool that is illustrated allows the operator to change reamers easily without handling the reamers directly, preventing contamination and fumbling any of the tiny reamer tools.

### BRIEF DESCRIPTION OF THE DRAWINGS

FIG. 1 is a side elevation of a vasectomy tool, with forceps.

FIG. 2 is an exploded side elevation of the vasectomy tool of FIG. 1.

FIG. 3a is a side elevation of a reamer holder/installation tool.

FIG. 3b is a schematic partial side elevation of a vasectomy tool, before the reamer is installed.

FIG. 4 is a schematic partial side elevation of the vasectomy tool of FIG. 3b, with the reamer installed in the retracted position.

FIG. 5 is a schematic partial side elevation of the vasectomy tool of FIG. 4, with the reamer in the extended position.

FIG. 6 is a schematic partial side elevation of the vasectomy tool of FIG. 5 with the reamer in the extended position, and about to enter the vas.

FIG. 7 is a schematic partial side elevation of the vasectomy tool of FIG. 6 with the reamer in the extended position, after the reamer is inserted into the vas.

FIG. 8 is a schematic partial side elevation of the vasectomy tool of FIG. 7 with the reamer in the extended position in the vas, and the forceps jaws closed upon the vas around the extended reamer.

FIG. 9 is a schematic partial side elevation of the vasectomy tool of FIG. 8 with the forceps fully compressed to release the catch and showing the reamer snapped back into the retracted position and out of the vas.

FIGS. 10-12 are schematic partial side elevations of various reamer cutting disk embodiments.

### BEST MODE OF CARRYING OUT THE INVENTION

Turning now to the drawings, the invention will be described in preferred embodiments by reference to the numerals of the drawing figures wherein like numbers indicate like parts.

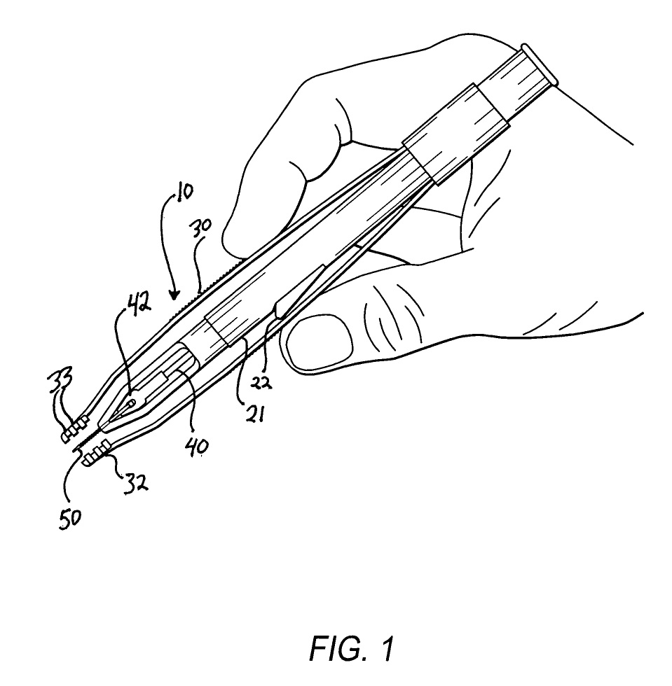

Reamer 50 illustrated in FIGS. 1-9 is advantageously made of stainless steel on a CNC microlathe. Alternatively, reamer 50 shown in FIG. 12 can be micro-injection molded from a hard polymer such as nylon, for reduced manufacturing cost and significantly reduced product cost with greater quantity availability in shorter time, with less tooling. This plastic part desirably eliminates the more complex back-cutting of other embodiments in favor of a simple planar back surface, which enhances removability from the injection mold.

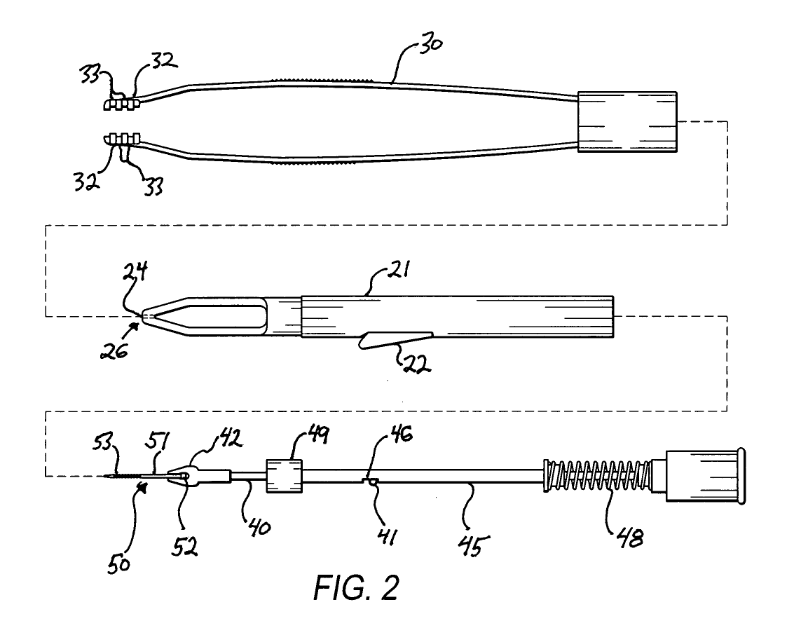

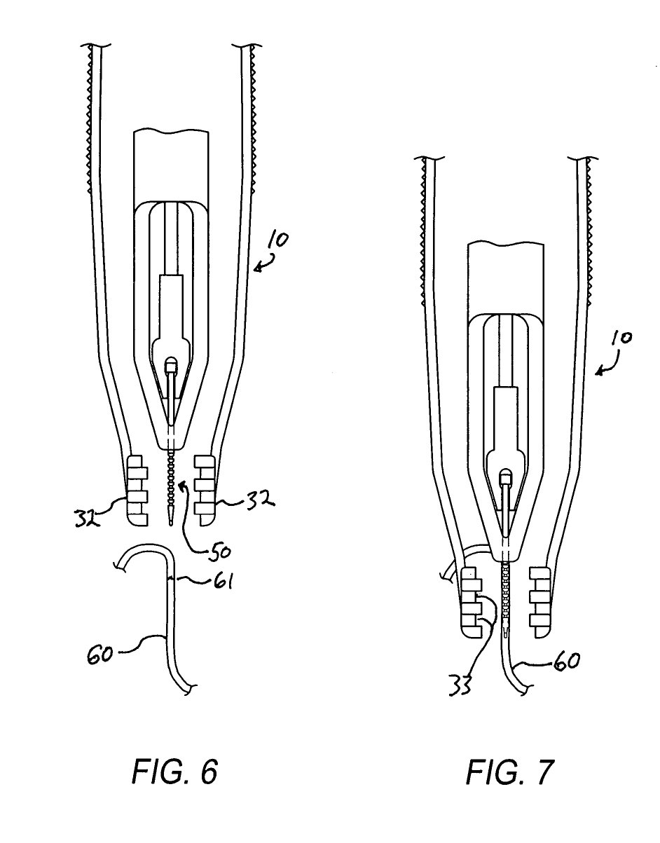

The spring motivated extractor embodiment of vasectomy tool 10 in FIG. 1 is generally in the shape of a retractable ball point pen, whose generally tubular handle or body 21 holds the compression spring, the pull rod to which the reamer is attached, and a catch mechanism which releasably keeps the compressed rod and spring in the extended and locked position. Button 22 on the side of the handle is in mating contact with catch release 41 (see FIG. 2), when catch release 41 is in locked position in pull rod casing 45, and pushing button 22 releases catch 41 to allow spring 48, rod 40 and reamer 50 to snap back, relative to handle 21, and out of vas 60 (see FIGS. 6-9).

The reusable embodiment of vas tool 10 uses a kind of flat two jaw collet 42 to hold reamer 50. This design facilitates side installation and removal of the reamer, which in turns facilitates easy removal of a tissue filled reamer, after extraction from the vas, and replacement of it with a fresh reamer for each successive vas segment to be reamed. Other reamer attachment methods may be employed, such as screw threads or clamps. A low cost, single-use, injection molded embodiment of the tool (not shown) functions roughly as described above, with the reamer and pull rod being a single unit, either by conventionally fusing or snap fitting a plastic reamer to a pull rod, or molding the reamer and pull rod as a unit. Such a single use embodiment would thus require 4 units per typical patient.

In FIG. 1 vas tool 10 with preferred forceps 30 is held in an operator's hand in the preferred manner of use. Forceps 30 has a pair of generally hemi-cylindrical hollow jaws 32, each of which is preferably equipped with overlapping and complimentary notches 33 that mesh when the jaws are fully closed. Alternate embodiments may dispense with these notches, and may employ alternate jaw shapes without departing from the scope of the invention. Tool 10 has handle 21 housing pull bar or pull rod 40 and its retraction mechanism (see FIG. 2), which is activated by button 22. Pull rod 40 has collet 42 into which reamer 50 is removably engaged and shown in the extended and locked position with reamer 50 protruding through aperture 24 at the tip of body 21.

In FIG. 2 an exploded assembly of tool 10 as shown in FIG. 1 is illustrated schematically. This detail of the retraction mechanism includes pull rod 40, rod casing 45, notch 46 in casing 45 for receiving in releasable locked relationship catch 41 to hold spring 48 in tension, all as will be appreciated by those skilled in the art, casing stop 49. Reamer 50 is shown with shaft 51, chuck end 52 engaged in pull rod collet 42, and cutting disk array 53. Body 21 is equipped with tissue stop 26, through which, via aperture 24, reamer 50 extends when extended and locked.

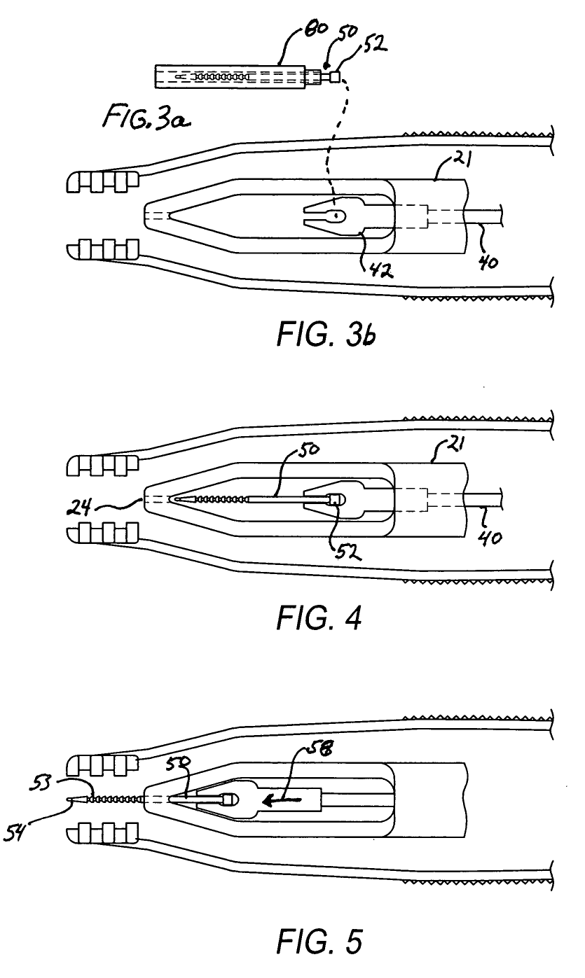

In FIGS. 3a, 3b and 4-5, reamer holder tool 80 is shown holding a reamer 50 with exposed chuck end 52, which is fitted sideways into the opening in collet 42 as shown in FIG. 3b with pull rod 40 in retracted position, and the tool 80 is simply slid off, leaving tool 10 as shown in FIG. 4. This procedure is then reversed to pick up the retracted and tissue filled reamer. Pull rod 40 is then cocked or extended in the direction of arrow 58 into the locked and ready position shown in FIG. 5.

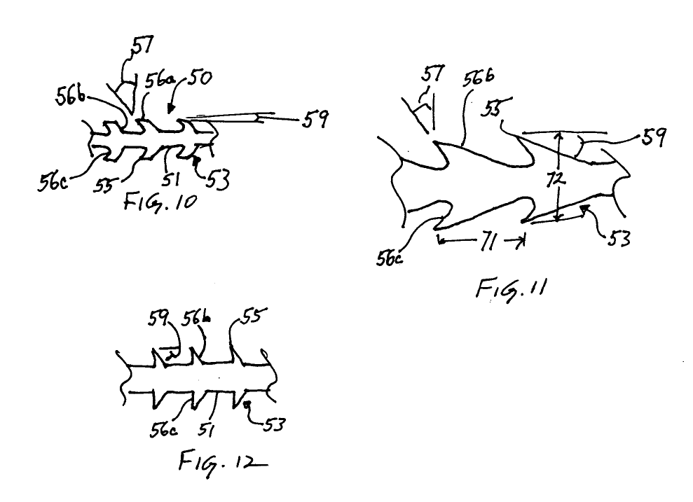

In FIGS. 10-12, various reamer 50 cutting disk 53 configurations are shown. In FIG. 10, cutting disks 53 are arrayed and spaced on shaft 51 of reamer 50. Each disk has a generally circumferential cutting edge 55. This cutting edge is generally formed by the intersection of back surface 56c and either leading surface 56b (for FIGS. 11 & 12), or back surface 56c and circumferential surface 56a (for FIG. 10). In preferred embodiments, angle 57 of back surface to shaft is around 45 degrees for FIG. 10. In FIG. 11, back surface 56c is disposed at an angle 57 of about 45 degrees to the axis of shaft 51, while frustoconical leading surface 56b is disposed at an angle 59 of about 20 degrees. In FIG. 12, back surface 56c is planar and generally normal to or about 90 degrees to the axis of shaft 51 and leading surface 56b is at an angle 59 of about 61 to 66 degrees and preferably about 63½ degrees, leaving a reciprocal blade angle of about 26½ degrees.

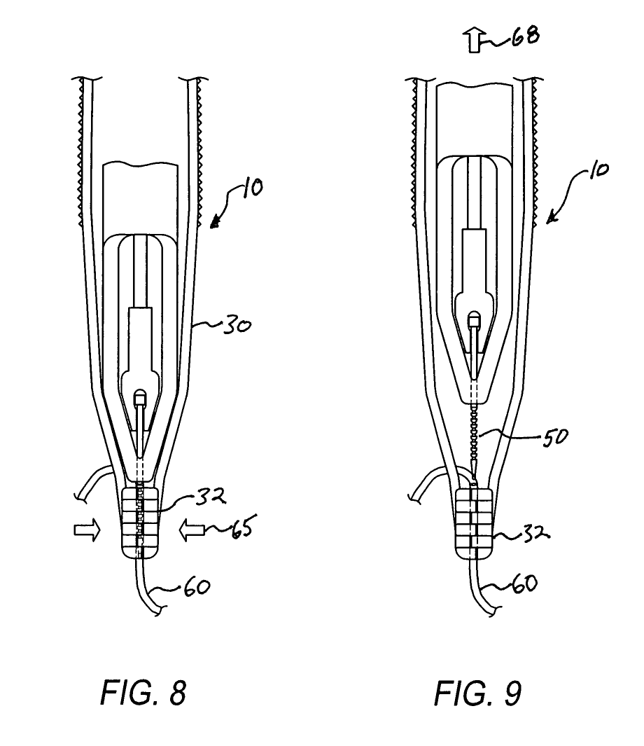

In FIGS. 6-9, the method proceeds generally along some or all of the following steps:
Vas 60 is exposed and lifted to allow partial transection 61 (FIG. 6) and insertion of probe-shaped reamer 50 into the vas lumen (FIG. 7).
Handle 21 is held parallel and generally coaxial to the long axis of exposed vas 60 during the procedure. Forceps 30 optional attachment to the tool's handle 21 is compressed in the direction of arrows 65 to bring forceps jaws 32 into compression against the vas tissue where it is adjacent to the inserted reamer (FIG. 8). When button 22 (not shown in FIG. 9; see FIG. 1) on handle 21 is pushed, preferably by further compression of the forceps against the button, which also meshes optional jaw notches 33, the reamer snaps back and out of the vas in the direction shown by arrow 68 (FIG. 9).
The filled reamer is removed from the holder and replaced with a fresh one, preferably without touching the reamers, such as by use of the illustrated cylindrical reamer holder and installation tool 80 (FIG. 3a).
Pull rod 40, now protruding from the rear end of the handle (not shown) is again compressed to compress spring 48 and engage catch 41 once again, to reset the spring release mechanism.
The procedure is repeated on the other half of the vas, and again on the opposite side vas. The small vasal segment between the reamer insertion points can either be left alone, crushed, removed or also reamed as described above.

Scarification of the distal and proximal ends of the partially transected vas is believed to be effective because the regenerative epithelial basement membrane of the vas is completely removed at the reamed site without harming vasal blood supply. Surgical dissection, tissue trauma and discomfort are reduced, as well as the chance of complications such as bleeding or formation of a sperm granuloma.

With regard to systems and components above referred to, but not otherwise specified or described in detail herein, the workings and specifications of such systems and components and the manner in which they may be made or assembled or used, both cooperatively with each other and with the other elements of the invention described herein to effect the purposes herein disclosed, are all believed to be well within the knowledge of those skilled in the art. No concerted attempt to repeat here what is generally known to the artisan has therefore been made.

### INDUSTRIAL APPLICABILITY

What is presented is a method and tool that can be used worldwide that is simple with minimal tissue dissection, and can be used in areas where finances and or power supply irregularities do not permit electro cautery. It is a device that can be either disposable, single use, or resterilizable, with readily available pre-sterilized kits.

In compliance with the statute, the invention has been described in language more or less specific as to structural features. It is to be understood, however, that the invention is not limited to the specific features shown, since the means and construction shown comprise preferred forms of putting the invention into effect. The invention is, therefore, claimed in any of its forms or modifications within the legitimate and valid scope of the appended claims, appropriately interpreted in accordance with the doctrine of equivalents.

We claim:

1. A vasectomy tool comprising a reamer operatively engaged upon a pull rod, the reamer further comprising a series of cutting disks on a shaft, the shaft having a blunt conical tip, a plurality of the cutting disks each having a generally circumferential cutting edge.

2. The tool of claim 1 wherein a plurality of the cutting disks have a generally frustoconical leading surface.

3. The tool of claim 2 wherein a plurality of the cutting disks have a generally back-cut back surface, the frusto conical leading surface and the back-cut back surface defining at least in part the circumferential cutting edge.

4. The tool of claim 3 wherein a plurality of the cutting disks have a generally straight back surface, the frusto conical leading surface and the straight back surface defining at least in part the circumferential cutting edge.

5. The tool of claim 1 wherein the reamer is removably engaged upon the pull rod.

6. The tool of claim 5 wherein the pull rod terminates in a collet, and the reamer terminates, at an end opposite the conical tip, in a chuck end sized to releasably engage the collet.

7. A vasectomy tool comprising a reamer operatively engaged upon a pull rod, the pull rod extendable into a releasably locked position against spring tension from a spring, the reamer further comprising a series of cutting disks on a shaft, the shaft having a blunt conical tip, a plurality of the cutting disks each having a generally circumferential cutting edge.

8. The tool of claim 7 wherein the tool further comprises a handle, the extendable pull rod and reamer disposed within the handle, the handle having a catch release button interengageable with a spring catch release on the pull rod.

9. The tool of claim 8 wherein the handle further comprises an integral forceps mechanism disposed on an outside of the handle, and operable therewith.

10. The tool of claim 7 wherein the reamer is removably engaged upon the pull rod.

11. The tool of claim 10 wherein the pull rod terminates in a collet, and the reamer terminates, at an end opposite the conical tip, in a chuck end sized to releasably engage the collet.

12. The tool of claim 7 wherein a plurality of the cutting disks a spaced from one another along the shaft at a distance approximately the same as a diameter of at least one disk.

13. The tool of claim 7 wherein a plurality of the cutting disks a spaced from one another along the shaft at a distance between 60% and 110% of a diameter of at least one disk.

14. The tool of claim 13 wherein a plurality of the cutting disks a spaced from one another along the shaft at a distance approximately 90% of a diameter of at least one disk.

15. The tool of claim 14 wherein a plurality of the cutting disks are approximately 1.1 millimeters in diameter and spaced from one another by approximately 1 millimeter.

16. The tool of claim 13 wherein a plurality of the cutting disks a spaced from one another along the shaft at a distance approximately ⅔ of a diameter of at least one disk.

17. A vasectomy tool comprising a reamer operatively engaged upon a pull rod, the pull rod extendable into a releasably locked position against spring tension from a spring, the reamer further comprising a series of cutting disks on a shaft, the shaft having a blunt conical tip, a plurality of the cutting disks each having a generally circumferential cutting edge, the tool further comprising a handle, the extendable pull rod and reamer disposed within the handle, the handle having a catch release button interengageable with a spring catch release on the pull rod, the handle comprising an integral forceps mechanism disposed on an outside of the handle, and operable therewith.

18. The tool of claim 17 wherein the reamer is removably engaged upon the pull rod.

19. The tool of claim 18 wherein the pull rod terminates in a collet, and the reamer terminates, at an end opposite the conical tip, in a chuck end sized to releasably engage the collet.

20. The tool of claim 17 wherein the forceps terminates in a pair of generally hemi-cylindrical jaws, the jaws extending past a body of the handle so that they are disposed to grip and overlap a vas section around the reamer when it is an extended and locked position.

21. The tool of claim 20 wherein the forceps jaws each have notches that are complimentary and overlapping with respect one jaw to the other.

22. A vasectomy method, the method comprising the steps of:
1. into a vas opening, generally sliding a multi-bladed reamer;
2. holding the vas against the inserted reamer;
3. quickly pulling the reamer out of the vas to remove at least a portion of vas mucosal layer and some of the vas muscle layer tissue;
whereby scarification of the vas is induced, and the vas is sealed by resultant scarring.

23. The method of claim 22 wherein the reamer comprises a series of cutting disks on a shaft, the shaft having a blunt conical tip, a plurality of the cutting disks each having a generally circumferential cutting edge.

24. The method of claim 22 wherein the reamer is sized to dilate the vas into which it is inserted.

25. The method of claim 24 wherein the reamer comprises a series of cutting disks on a shaft, the shaft having a blunt conical tip, a plurality of the cutting disks each having a generally circumferential cutting edge.

26. The method of claim 25 wherein the cutting edges of a plurality of the cutting disks are adapted for tearing rather than cutting tissue as the reamer is removed quickly.

27. The method of claim 22 wherein the reamer comprises a spring-loaded pull bar, and the pull bar and attached reamer are pulled out of the vas under selectable spring tension.

28. The method of claim 27 wherein the reamer and pull bar are disposed within a spring-loaded handle that includes a vas-gripping forceps mechanism, and the step of holding the vas is accomplished with the forceps, and the step of quickly pulling out the reamer is accomplished with the release of the spring-loaded pull bar.

29. The method of claim 27 wherein the reamer is removably attachable to the pull bar, and before and after the steps of sliding in the reamer and quickly removing the reamer, a removable reamer is respectively attached to the pull bar, and removed from the pull bar.

30. The method of claim 29 wherein the steps of attaching and removing the removable reamer are effected with a holder tool for inserting and removing the reamer into and out of the pull bar.
